(19) United States
(12) Patent Application Publication
(10) Pub. No.: US 2024/0358231 A1
(43) Pub. Date: Oct. 31, 2024

(54) DUAL-LOCKING MEDICAL DEVICES AND RELATED METHODS

(71) Applicant: Boston Scientific Scimed, Inc., Maple Grove, MN (US)

(72) Inventor: Sean POWELL, Holden, MA (US)

(73) Assignee: Boston Scientific Scimed, Inc., Maple Grove, MN (US)

(21) Appl. No.: 18/645,595

(22) Filed: Apr. 25, 2024

Related U.S. Application Data
(60) Provisional application No. 63/498,294, filed on Apr. 26, 2023.

(51) Int. Cl. A61B 1/00; A61B 1/018

(52) U.S. Cl. CPC A61B 1/00098; A61B 1/00128; A61B 1/018

(57) ABSTRACT

A distal tip of a medical device may comprise: a housing and an elevator. The elevator may include an opening extending from a first surface of the elevator to a second surface of the elevator. The opening may be configured to receive an accessory medical device therethrough, such that the accessory medical device extends distally of a distalmost end of the housing.

### CROSS-REFERENCE TO RELATED APPLICATIONS

This application claims the benefit of priority of U.S. Provisional Patent Application No. 63/498,294, filed on Apr. 26, 2023, the entirety of which is incorporated herein by reference.

### TECHNICAL FIELD

Various aspects of this disclosure relate generally to dual-locking medical devices and related methods. In particular, aspects of this disclosure pertain to elevators and distal tip features of duodenoscopes, among other aspects.

### BACKGROUND

Duodenoscopes may include a handle and a sheath, and the sheath may be insertable into a body lumen of a subject. The sheath may terminate in a distal tip portion, which may include features such as optical elements (e.g., camera, lighting), air/water outlets, and working channel openings. An elevator may be disposed at a distal tip and may be actuatable in order to change an orientation of a medical device/tool passed through the working channel. For example, the elevator may be pivotable or otherwise movable.

Elements in the handle may control the elements of the distal tip. For example, buttons, knobs, levers, etc. may control elements of the distal tip. The elevator may be controlled via a control mechanism in a handle, such as a lever, which may be attached to a control wire that attaches to the elevator. When an actuator (e.g., a lever) is actuated, the wire may move proximally and/or distally, thereby raising and/or lowering the elevator. The elevator may lock an instrument inserted through a working channel of the duodenoscope in a retroflexed configuration of the instrument or other configuration in which the instrument extends radially outward, but not in other configurations of the instrument. Therefore, a need exists for dual-locking medical devices and related methods.

### SUMMARY

Each of the aspects disclosed herein may include one or more of the features described in connection with any of the other disclosed aspects.

In an example, a distal tip of a medical device may comprise: a housing and an elevator. The elevator may include an opening extending from a first surface of the elevator to a second surface of the elevator. The opening may be configured to receive an accessory medical device therethrough, such that the accessory medical device extends distally of a distalmost end of the housing.

Any of the exemplary devices or methods herein may include any of the following features. The first surface of the elevator may be on a first side of an axis of rotation of the elevator. The second surface of the elevator may be on a second side of the axis of rotation. The first side may be opposite the second side. In a raised configuration of the elevator, the opening may configured to receive the accessory medical device therethrough. In the raised configuration of the elevator, the first surface may be proximal of the second surface. The housing may include a boss. In a lowered configuration of the elevator, the boss may be configured to be received within the opening. The second surface of the elevator and the boss may be configured to engage the accessory medical device in a configuration in which the accessory medical device is received through the opening, thereby retaining the accessory medical device in a locked configuration, in which the accessory medical device extends approximately parallel to a longitudinal axis of the distal tip. The boss may include a depression. The depression may be configured to receive the accessory medical device. The second surface of the elevator may include a recess. In the locked configuration, the accessory medical device may be received within a recess of the second surface. The recess may be distal of the opening. The opening may have a fully enclosed perimeter. The opening may be approximately circular. The distal tip also may include a channel. The elevator may be received within the channel. The channel may have an open distal end. The housing may define at least one surface of the channel. The at least one surface may include a notch at a distal end. The housing may define a locking surface at a proximal end of the channel, wherein the locking surface is configured to mate with the first surface of the elevator in a raised configuration of the elevator. In a lowered configuration of the elevator, the distal tip may be configured to receive the accessory medical device such that the accessory medical device extends from a proximal end of the elevator to a distal end of the elevator.

In another example, an elevator of a medical device may include an axle. The elevator may be rotatable about the axle. An axis of rotation of the elevator may extend through the axle. The elevator also may include first surface on a first side of the axis of rotation; a second surface on a second side of the axis of rotation; and an opening extending through the elevator, between the first surface and the second surface.

Any of the devices disclosed herein may have an opening with a fully enclosed perimeter.

In an aspect, a method of using a medical device may comprise: inserting an accessory medical device into a working channel of the medical device; extending the accessory medical device distally to a distalmost end of the medical device, with an elevator of the medical device in a raised configuration; and using the accessory medical device to perform a medical procedure.

Any of the aspects disclosed herein may have any of the following features. After extending the accessory medical device distally, the method may include at least partially lowering the elevator to engage the elevator between a distal surface of the elevator and a surface of a distal tip of the medical device. The surface of the distal tip may be a surface of a boss extending from a housing of the distal tip.

It may be understood that both the foregoing general description and the following detailed description are exemplary and explanatory only and are not restrictive of the invention, as claimed. As used herein, the terms “comprises,” “comprising,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “distal” refers to a direction away from an operator, and the term “proximal” refers to a direction toward an operator. The term “exemplary” is used in the sense of “example,” rather than “ideal.” The term “approximately,” or like terms (e.g., “substantially”), includes values +/−10% of a stated value.

### BRIEF DESCRIPTION OF THE DRAWINGS

The accompanying drawings, which are incorporated in and constitute a part of this specification, illustrate aspects this disclosure and together with the description, serve to explain the principles of the disclosure.

FIGS. 1A and 1B depict an exemplary medical device.

FIGS. 2A-2D depict a distal tip of the medical device of FIGS. 1A-1B with an elevator in a lowered position.

FIGS. 3A-3D depict the distal tip of the medical device of FIGS. 1A-1B with an elevator in a raised position.

FIGS. 4A-4D depict the distal tip of the medical device of FIGS. 1A-1B with a smaller diameter accessory device extending therethrough.

FIGS. 5A-5D depict the distal tip of the medical device of FIGS. 1A-1B with a larger diameter accessory device extending therethrough.

FIG. 6A-6E depict an elevator of the distal tip of the medical device of FIGS. 1A-5D.

FIGS. 7A-9B depict another distal tip with an accessory device extending therethrough.

FIGS. 10A-10B depict the distal tip of FIGS. 7A-9B without an elevator.

FIGS. 11A-11D depict an elevator of the distal tip of the medical device of FIGS. 7A-9B.

### DETAILED DESCRIPTION

Conventionally, operators may utilize different types of scope devices for different medical procedures. For example, upper endoscopy or esophagogastoduodenoscopy (“EGD”) may be performed using a forward-viewing endoscope. A forward viewing endoscope may include at its distal tip an imager and/or a working channel opening that are forward-facing (i.e., face distally). Other procedures, such as endoscopic retrograde cholangiopancreatography (“ERCP”) may be performed using a side-viewing duodenoscope. A side-viewing duodenoscope may include at its distal tip an imager and/or a working channel opening that are side-facing (e.g., face radially outward relative to a central longitudinal axis of a shaft of the duodenoscope).

A distal tip side-viewing medical device, such as a duodenoscope, may include an elevator. The elevator may be operatively connected to an actuator in a handle of the duodenoscope. When an operator activates the actuator, the elevator may be raised/lowered. As an operator inserts an accessory device (e.g., an instrument or a guidewire) into a working channel of the medical device and advances the instrument through the working channel, the elevator originally may be in a lowered configuration. After the accessory device has been extended out of a distal opening of the working channel, the elevator may be raised so as to deflect a distal tip of the accessory device. When deflected, the accessory device may have a retroflexed shape or other shape where the accessory device protrudes radially outward. The elevator may lock the accessory device in a retroflexed configuration or other configuration where the accessory device extends radially outward. Such deflection may be utilized to allow an operator to access a subject's biliary tract and/or to cannulate a subject's major papilla.

A conventional side-viewing medical device with a conventional elevator may be useful only for certain medical procedures. It may be desirable to have a medical device that is also configured to have a device extend distally therefrom, without being deflected. Such a medical device may be useful for both EGD and ECRP procedures. However, a conventional elevator may deflect an instrument in a lowered configuration, such that a device extends radially outward, in addition to extending distally from the working channel. In other words, such an elevator may partially articulate/deflect an accessory device even in a fully open/lowered configuration. Furthermore, a distal tip of a medical device with an elevator may be open on one side, which may allow an accessory device to move through the open side, unconstrained. Such an arrangement contrasts with, for example, a conventional distally facing medical device (e.g., an endoscope) with a working channel that opens on the distal face; such a working channel and distal tip arrangement prevents an accessory device from extensively moving in a radial direction.

The medical devices disclosed herein may include an elevator having a fenestration formed therein. When the elevator is in a lowered position, a boss of a housing of a distal tip of a medical device may be received within the fenestration. When an accessory device is passed through a working channel of the medical device with the elevator in a lowered position, the accessory device may extend from the distal tip in a manner similar to a conventional duodenoscope. The elevator may be actuated to deflect the accessory device and lock the accessory device in the raised position. Using the medical device in such a manner, an operator may, for example, perform medical procedures on a pancreatico-biliary tract of a subject, such as ERCP.

For procedures in which the operator desires to use the medical device more akin to a forward/distally-facing endoscope (i.e., with the accessory device extending distally from the medical device without being retroflexed or extending radially outward), the operator may raise the elevator to a raised position and then pass an accessory device through the working channel. The accessory device may extend through the fenestration, and extend distally from the medical device (e.g., similarly to how a device extends from a working channel of a forward-facing endoscope). The elevator and/or the boss may include features that may facilitate locking the accessory device in such a position (i.e., a position in which the accessory device extends distally, with its longitudinal axis approximately parallel to or coaxial with a longitudinal axis of the medical device). Thus, the medical device, including the fenestrated elevator, may allow for dual or two-fold locking—locking accessory devices in an articulated/retroflexed/radially-outward configuration or in a straight/distally extending configuration.

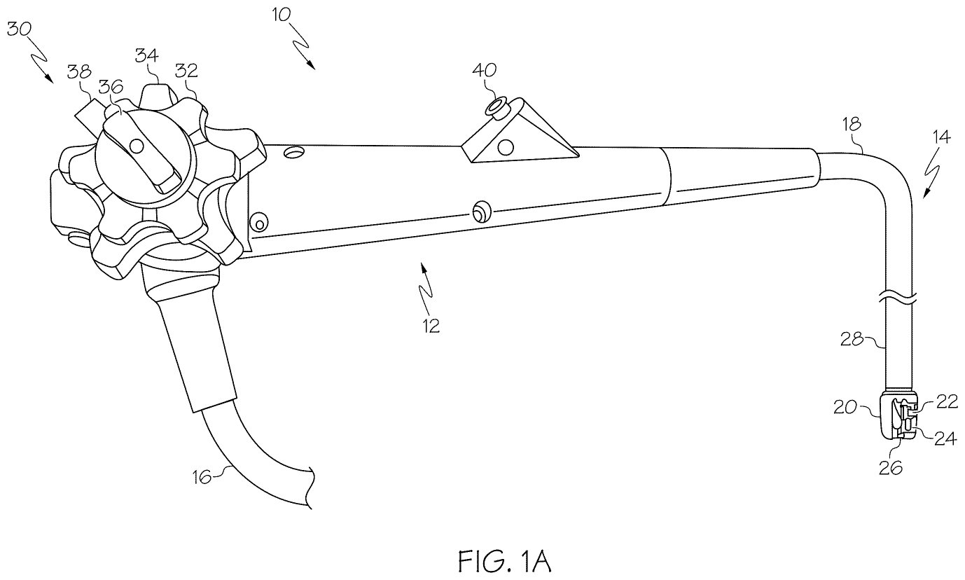

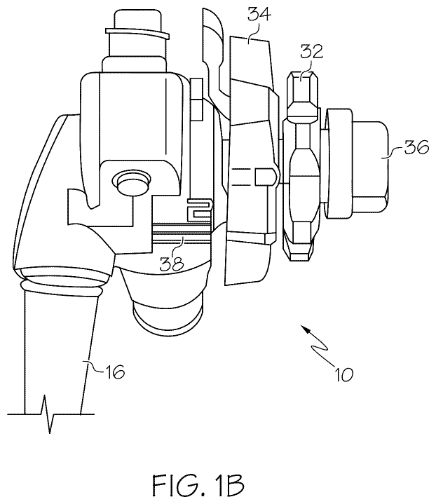

FIG. 1A depicts an exemplary medical device 10 having a handle 12 and an insertion portion 14. FIG. 1B shows a proximal end of handle 12. Medical device 10 may also include an umbilicus 16 for purposes of connecting medical device 10 to sources of, for example, air, water, suction, power, etc., as well as to image processing and/or viewing equipment. Although duodenoscopes and endoscopes (and combination devices that perform functions of duodenoscopes and endoscopes) are particularly referenced herein, the disclosure also encompasses other types of devices, such as bronchoscopes, gastroscopes, endoscopic ultrasound (“EUS”) scopes, colonoscopes, ureteroscopes, bronchoscopes, laparoscopes, cystoscopes, aspiration scopes, sheaths, catheters, or similar devices. A reference to a duodenoscope herein should be understood to encompass any of the above medical devices.

Insertion portion 14 may include a sheath or shaft 18 and a distal tip 20. Distal tip 20 may include an imaging device 22 (e.g., a camera) and a lighting source 24 (e.g., an LED or an optical fiber). Distal tip 20 may be side-facing. That is, imaging device 22 and lighting source 24 may face radially outward, perpendicularly, approximately perpendicularly, or otherwise transverse to a longitudinal axis of shaft 18 and distal tip 20. Additionally or alternatively, distal tip 20 may include one or more imaging devices 22 that face in more than one direction. For example, a first imaging device 22 may face radially outward, and a second imaging device 22 may face distally (approximately parallel to a longitudinal axis of distal tip 20/shaft 18).

Distal tip 20 may also include an elevator 26 for changing an orientation of an accessory device or a tool inserted in a working channel of medical device 10. Elevator 26 may alternatively be referred to as a swing stand, pivot stand, raising base, or any suitable other term. Elevator 26 may be pivotable via, e.g., an actuation wire or another control element that extends from handle 12, through shaft 18, to elevator 26.

A distal portion of shaft 18 that is connected to distal tip 20 may have a steerable section 28. Steerable section 28 may be, for example, an articulation joint. Shaft 18 and steerable section 28 may include a variety of structures which are known or may become known in the art.

Handle 12 may have one or more actuators/control mechanisms 30. One or more of control mechanisms 30 may provide control over steerable section 28. One or more of control mechanisms may allow for provision of air, water, suction, etc. For example, handle 12 may include control knobs 32, 34 for left, right, up, and/or down control of steerable section 28. For example, one of knobs 32, 34 may provide left/right control of steerable section 28, and the other of knobs 32, 34 may provide up/down control of steerable section 28. Handle 12 may further include one or more locking mechanisms 36 (e.g., knobs or levers) for preventing steering and/or braking of steerable section 28 in at least one of an up, down, left, or right direction. Handle 12 may include an elevator control lever 38 (see FIG. 1B). Elevator control lever 38 may raise and/or lower elevator 26, via connection between lever 38 and an actuating wire that extends from lever 38, through shaft 18, to elevator 26. A port 40 may allow passage of a tool through port 40, into a working channel of the medical device 10, through shaft 18, to distal tip 20.

In use, an operator may insert at least a portion of shaft 18 into a body lumen of a subject. Distal tip 20 may be navigated to a procedure site in the body lumen. The operator may insert an accessory device (not shown) into port 40, and pass the accessory device through shaft 18 via a working channel to distal tip 20. The accessory device may exit the working channel at distal tip 20. The user may use elevator control lever 38 to raise elevator 26 and angle the accessory device toward a desired location (e.g., a papilla of the pancreatico-biliary tract). The user may use the accessory device to perform a medical procedure.

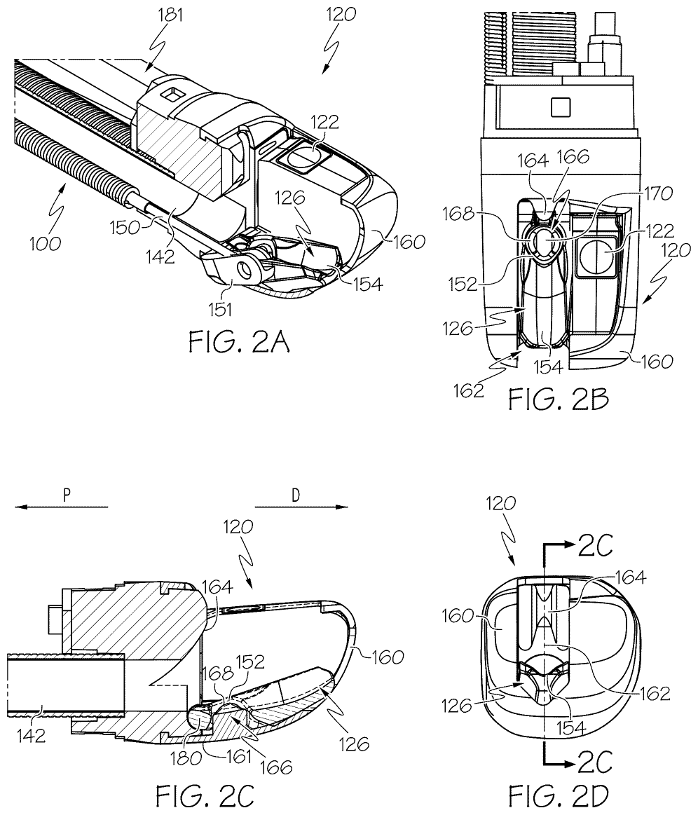

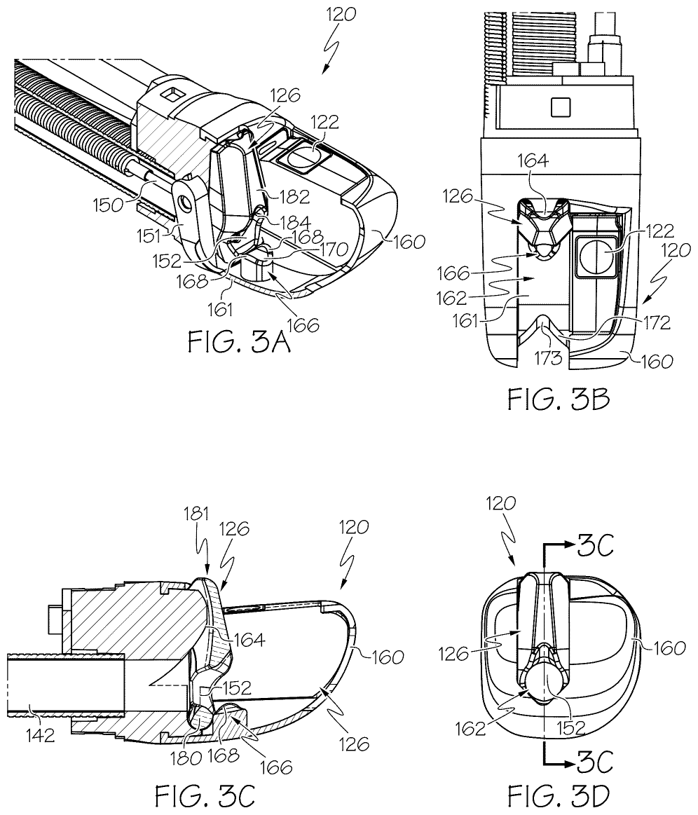

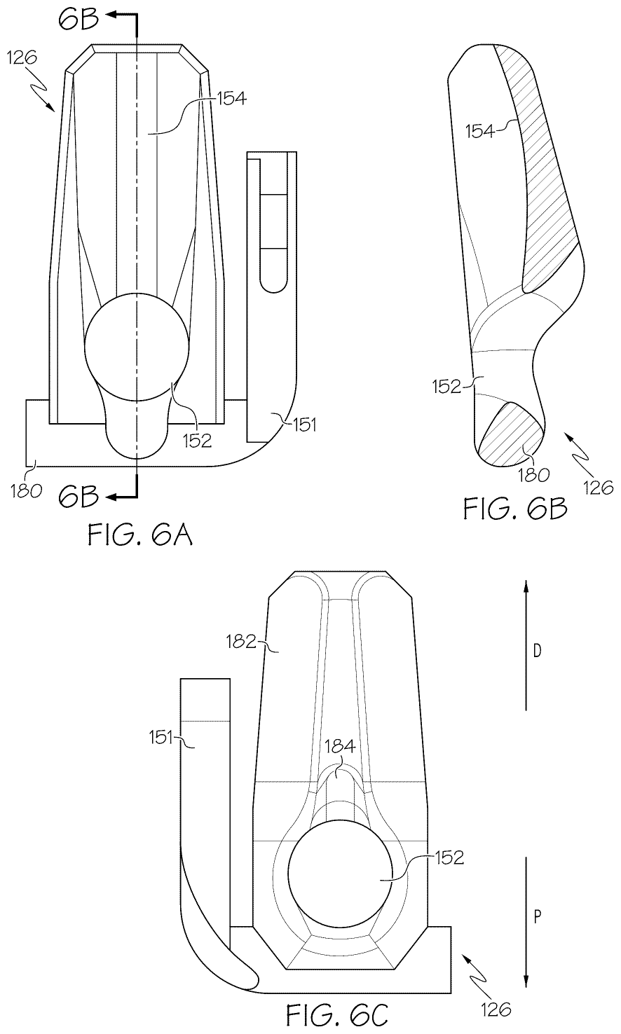

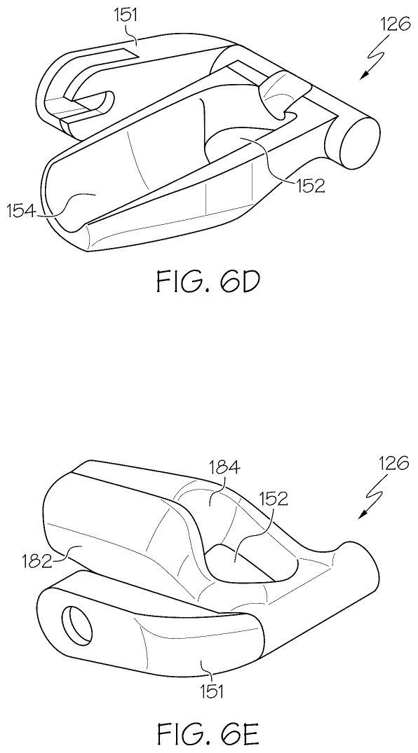

FIGS. 2A-5D depict a distal tip 120, for use with medical device 10, in various configurations. Distal tip 120 may have any of the features of distal tip 20. FIGS. 2A, 3A, 4A, and 5A depict perspective cross-sections of distal tip 120 in various configurations, discussed below. FIGS. 20, 3C, 4C, and 5C illustrate cross-sections taken along the respectively labeled lines in FIGS. 2D, 3D, 4D, and 5D. Certain elements of distal tip 120 (e.g., caps, covers, sheaths, etc.) may be omitted from FIGS. 2A-5D to more clearly illustrate aspects of the disclosure. FIGS. 6A-6E depict aspects of an elevator 126 of distal tip 120. FIG. 6B illustrates a cross-section taken along line 6B of FIG. 6A. In certain figures, arrows labeled “P” and “D” denote proximal and distal directions, respectively. Elevator 126 may have any of the features of elevator 26. Where feasible, FIGS. 2A-6E denote corresponding elements from FIGS. 1A-1B by adding 100 to the reference numbers of FIGS. 1A-1B. Although some reference labels associated with distal tip 120 or elevator 126 are omitted from certain figures for ease of illustration, it will be appreciated that the structures of distal tip 120 described herein pertain to each of FIGS. 2A-5B where the structures are visible, and the aspects of elevator 126 described herein apply to each of FIGS. 2A-6E where the aspects are visible.

Distal tip 120 may include a housing 160 for receiving the components described herein. Housing 160 may be formed from any suitable material (e.g., metal or plastic) and may have any suitable shape. In some examples, housing 160 may be hollow and may receive components of distal tip 120 therein or thereon. Housing 160 may be constructed according to any suitable process (e.g., molding or additive manufacturing). Housing 160 may include a cap that fits over other elements of distal tip 120.

Distal tip 120 may include an imaging device 122 (shown in, for example, FIGS. 2A and 2B). Although imaging device 122 is depicted as side-viewing, it will be appreciated that distal tip 120 also or alternatively may include one or more forward (i.e., distally) facing imaging devices. Although a lighting source, such as lighting source 24, is not shown in FIGS. 2A-5D, it will be appreciated that distal tip 120 may include one or more lighting sources having desired features (such as any of the features of lighting source 24) and pointing in any desired direction. As shown in, for example, FIGS. 2A and 2C, a working channel 142 may extend through shaft 18 to an opening in distal tip 120. Working channel 142 may have any suitable dimensions and may be used to receive, for example, one or more accessory devices passed through port 40 of handle 12.

Elevator 126 may be received within a channel 162 (see, e.g., FIGS. 2B, 2D, 3B, 4B, and 5B) of distal tip 120. Channel 162 may extend approximately parallel to a central longitudinal axis of distal tip 120/shaft 18. Channel 162 may be in communication with working channel 142. Channel 162 may be open on two sides (e.g., a top side adjacent to imaging device 122 and a distal side), in addition to being in communication with a distal opening of working channel 142. As used herein, the terms “top,” “up,” “upward,” and the like refer to an upward direction of FIGS. 2C-2D, 3C-3D, 4C-4D, and 5C-5D. The terms “bottom,” “down,” “downward,” and the like refer to a downward direction of FIGS. 2C-2D, 3C-3D, 4C-4D, and 5C-5D. As compared with a conventional duodenoscope, channel 162 may be elongated along a proximal/distal direction and open on the distal side of channel 162.

Elevator 126 may be coupled to a control wire 150 via an arm 151 of elevator 126. Arm 151 is merely an exemplary structure for coupling control wire 150 to elevator 126, and control wire 150 may be coupled to elevator 126 in any suitable manner. Control wire 150 may be directly or indirectly coupled to lever 38 (see FIG. 1B, described above). As lever 38 is rotated or otherwise actuated, control wire 150 may be moved proximally or distally. As lever 38 is rotated in a first direction, control wire 150 may move proximally, thereby raising elevator 126 to a raised configuration (such as a fully raised configuration of elevator 126, as shown in FIGS. 3A-3D and described in further detail below). As lever 38 is rotated in a second direction (i.e., opposite to the first direction), control wire 150 may move distally, thereby lowering elevator 126 to a lowered configuration (e.g., to a fully lowered configuration as shown in FIGS. 2A-2D and described in further detail below). Elevator 126 may have various intermediate positions between the fully raised configuration and the fully lowered configuration. Elevator 126 may rotate about an axle 180 at a proximal end of elevator 126. An axis of rotation of elevator 126 may extend through axle 180.

Elevator 126 may include a first, guide surface 154 (see, e.g., FIGS. 2A and 2B), which extends between axle 180 of elevator 126 and a distal end 181 (e.g., a distalmost end) of elevator 126 (see FIGS. 2C and 3C). Elevator 126 also may include a second, back surface 182 extending between axle 180 and distal end 181, on an opposite side of elevator 126 from guide surface 154. Guide surface 154 may be on a first side of the axis of rotation of elevator 126, and back surface 182 may be on a second side (opposite the first side) of the axis of rotation of elevator 126. When elevator 126 is in a lowered configuration (see FIGS. 2A-2D), guide surface 154 may face an open side of channel 162. In the lowered configuration of FIGS. 2A-2D, back surface 182 may face a surface of housing 160. When elevator 126 is in a fully raised configuration (see FIGS. 3A-3D), guide surface 154 may be a proximal side of elevator 126, and back surface 182 may be a distal side of elevator 126.

As shown in, for example, FIGS. 2B, 2C, 3A, 3C, 3D, 4C, 4B, 5A, 50, 5D, and 6A-6E, elevator 126 may include a fenestration 152. Fenestration 152 may extend through an entire thickness of elevator 126, between guide surface 154 and back surface 182. Fenestration 152 may have any suitable shape. For example, fenestration 152 may define an approximately round (circular) opening, as shown particularly in FIG. 6A and 6C. A diameter/width of an opening defined by fenestration 152 may be any suitable size (e.g., approximately the same size or larger than a diameter/width of a largest accessory device to be used with device 10). In some examples, fenestration 152 may have a diameter/width that is equal to or smaller than a diameter/width of working channel 142. A size of fenestration 152 may depend upon a width of elevator 126. Elevator 126 needs to have sufficient wall strength on at least one side of fenestration 152 to apply a frictional force when elevator 126 is fully raised or fully lowered. For example, fenestration 152 may be approximately 3.5 mm (which may be smaller than a size of working channel 142, which may be approximately 4.2 mm) or may have any suitable size (e.g., any size less than or equal to approximately 6.35 mm (0.25 inches), and working channel 142 may have a similar size, e.g., less than or equal to approximately 6.35 mm (or 0.25 inches). Fenestration 152 may be configured to receive an accessory device therethrough. Although fenestration 152 is depicted as a hole, it will be appreciated that fenestration 152 alternatively may be any type of slot, notch, gap, recess, or other type of opening. Fenestration 152 may be fully enclosed around a perimeter/circumference of fenestration 152 (i.e., a hole), as shown. Alternatively, fenestration 152 may include one or more open sides, such that fenestration 152 forms a slot or notch.

Guide surface 154 and/or back surface 182 may be contoured adjacent to fenestration 152 For example, as shown in FIG. 6C, back surface 182 may include a contoured recess 184 distally of and adjacent to fenestration 152. As described in detail below, contoured recess 184 may be shaped and sized so as to interface with an accessory device (e.g., a small-diameter accessory device). As shown in FIG. 6C, contoured recess 184 may taper distally in width (a horizontal direction in FIG. 6C) and in depth (a direction into/out of FIG. 6C), such that contoured recess 184 becomes narrower and shallower along the distal direction.

A bottom wall 161 (see, e.g., FIGS. 2C, 3A, and 3B) of housing 160 may define a bottom surface of channel 162. Bottom wall 161 may have a boss 166 disposed thereon. Boss 166 may be formed integrally (i.e., monolithically, from a same piece of material) with bottom wall 161 or may be formed separately from bottom wall 161 and attached thereto. As shown particularly in FIG. 2B, boss 166 may have an approximately round cross-sectional shape. Boss 166 alternatively may have any suitable shape. A shape and size of fenestration 152 may be complementary to a shape and size of boss 166, such that boss 166 may fit within/mate with fenestration 152. Fenestration 152 may have a slightly larger diameter/width than boss 166, to allow boss 166 and elevator 126 to freely move with respect to one another as elevator 126 is raised/lowered. As shown particularly in FIG. 3A, boss 166 may include two ridges 168 on either side thereof, with a depression 170 between the two ridges 168. Depression 170 may extend approximately parallel to a central longitudinal axis of distal tip 120/shaft 18.

The various configurations and uses of distal tip 120 will now be described, along with methods for using distal tip 120. In an example, device 10 and distal tip 120 may be used as a typical side-viewing medical device (e.g., for an ERCP procedure). Elevator 126 may have a first, lowered configuration, as shown in FIGS. 2A-2D. Boss 166 may be received within fenestration 152. Boss 166 may serve to plug fenestration 152. As shown in FIG. 2C, boss 166 may protrude from bottom wall 161 to a height that is approximately the same as a thickness of elevator 126 between guide surface 154 and back surface 182. A top surface of boss 166 may approximately align with guide surface 154. During an ERCP procedure or other procedure in which side-viewing features of device 10 are to be utilized, an accessory device (e.g., a guidewire) may be passed through working channel 142 with elevator 126 in the lowered configuration. With boss 166 received within fenestration 152, the accessory device may pass along the guide surface 154 as with a conventional elevator of a side-viewing scope, until a distal tip of the accessory device is distal of distal end 181 of elevator 126.

During an ERCP procedure, the user may raise elevator 126 to a raised position/configuration, as shown in FIG. 3A-3D, thereby articulating the accessory device (e.g., to a retroflexed configuration of the accessory device or another configuration where the accessory device extends radially outward (e.g., approximately 90 degrees outward). For ease of illustrating the features of distal tip 120, an accessory device is not depicted in FIGS. 2A-3D. In some aspects, the accessory device (e.g., guidewire) may be locked (retained in a desired position/configuration) between a portion of guide surface 154 (e.g., distal end 181) and a locking surface 164 of housing 160. Locking surface 164 may be at a proximal side of channel 162. Locking surface 164 may have a complementary shape to guide surface 154, such that guide surface 154 may mate with locking surface 164. Additionally or alternatively, locking surface may have an approximately “V” shape. Locking surface 164 may have any features known in the art. In the raised configuration, although boss 166 is no longer positioned within fenestration 152, a distal end of the accessory device has already been extended past distal end 181. Thus, fenestration 152 does not interfere with an articulation of the accessory device or with locking of the accessory device between guide surface 154 and locking surface 164. Thus, distal tip 120 allows a user to perform a procedure such as ECRP as it is conventionally performed with a side-viewing device.

In another example, a user may pass an accessory device over elevator 126 with elevator 126 in the lowered configuration/position, as described above. The user may perform a procedure without raising elevator 126. For example, the user may use medical device 10 as a forward-viewing device for, e.g., an EGD procedure. In such a configuration, the accessory device may be free to move (e.g., float) up/down through channel 162, including through the open top side of channel 162.

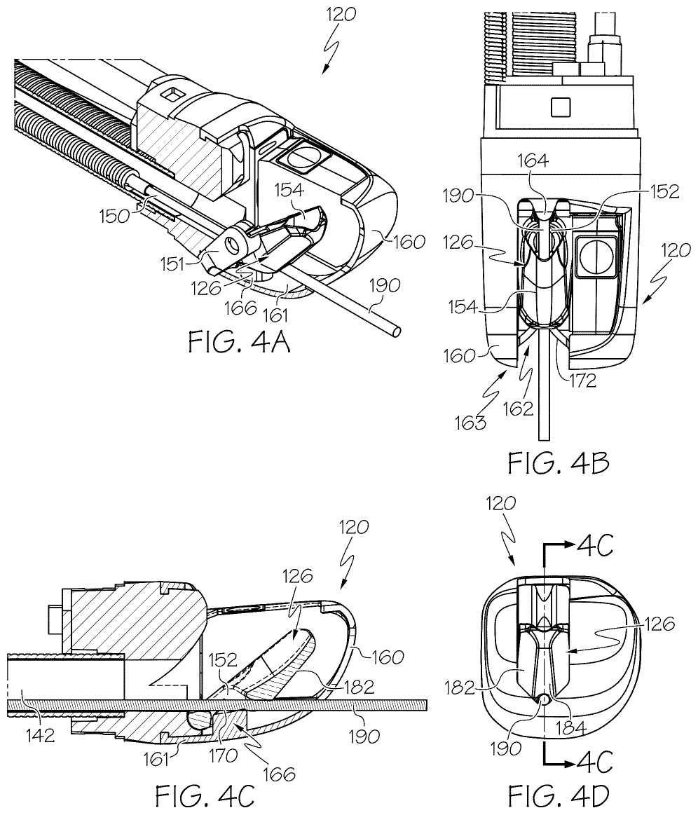

Alternatively, the user may desire to perform a procedure such as an EGD procedure with an accessory device in a locked position, unable to freely move upward/downward. In order to do so, before passing the accessory device fully through working channel 142, the user may raise elevator 126 to a raised position, as shown in FIGS. 3A-3D. The user may then extend the accessory device through working channel 142, and through fenestration 152 of elevator 126. Working channel 142 may be in communication with fenestration 152. For example, fenestration 152 may be approximately aligned with working channel 142. Features of guide surface 154 may guide the accessory device through fenestration 152. Fenestration 152 may be akin to a distal opening of working channel 142 when elevator 126 is in a fully raised configuration, as shown in FIGS. 3A-3D. The accessory device may extend distally of a distalmost end 163 (see FIGS. 4B and 4C) of housing 160 and a distalmost end of channel 162.

Distal tip 120 may be operative to retain (i.e., lock) both small-diameter and large-diameter accessory devices so that the accessory device extends distally of distal tip 120 (e.g., approximately parallel to a longitudinal axis of distal tip 120 and/or shaft 18). FIGS. 4A-4D depict a small-diameter accessory device 190, such as a guidewire, in a locked configuration. For example, small-diameter accessory device may have a diameter greater than or equal to 0.020 inches (e.g., greater than or equal to 0.025 inches). In FIG. 4A, a portion of small-diameter accessory device proximal of elevator 126 is partially obscured by control wire 150. As described above, small-diameter accessory device 190 may be passed through fenestration 152 with elevator 126 in a raised position. To lock small-diameter accessory device 190, a user may partially lower elevator 126, such that back surface 182 of elevator 126 and boss 166 engage small-diameter accessory device 190, as shown in FIGS. 4A-4D. This engagement may retain small-diameter accessory device 190 such that it extends approximately parallel to a longitudinal axis of distal tip 120/shaft 18 (i.e., lock small-diameter accessory device 190). An amount or degree that elevator 126 is lowered to lock small-diameter accessory device 190 may depend upon one or more dimensions or other properties of distal tip 120 and/or a diameter/width of small-diameter accessory device 190. Small-diameter accessory device 190 may extend through depression 170 in the locked configuration. Contoured recess 184 may have a shape that mates with/interacts with small-diameter accessory device 190 so as to engage small-diameter accessory device 190 between contoured recess 184 and a portion of boss 166, such as depression 170.

As shown in FIGS. 3B and 4B, a distal end of bottom wall 161 of housing 160 may have a notch 172, at a distal end of channel 162. Notch 172 may be approximately triangularly shaped, with a proximal apex 173 (FIG. 3B). When small-diameter accessory device 190 is in a locked configuration, as shown in FIGS. 4A-4D, small-diameter accessory device 190 may be received within notch 172, at apex 173. Apex 173 may further help to restrain small-diameter accessory device 190 so that small-diameter accessory device 190 extends distally, approximately parallel to a longitudinal axis of distal tip 120 and/or shaft 18. Additionally or alternatively, housing 160 may include a protrusion (not shown) similar to locking surface 164 to restrain small-diameter accessory device 190. Such a protrusion may be between notch 172 and depression 170 and may have a shape that complements a shape of contoured recess 184. To unlock small-diameter accessory device 190, elevator 126 may be raised. Small-diameter accessory device 190 may then be removed (e.g., proximally retracted) or repositioned.

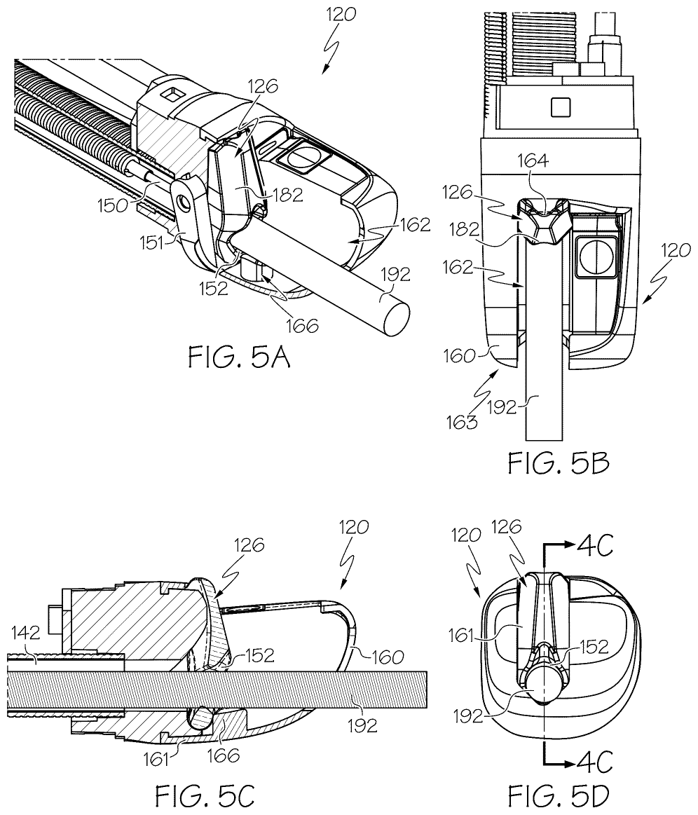

Distal tip 120 also may be utilized to position and retain a large-diameter accessory device 192, as shown in FIGS. 5A-5D. For example, large-diameter accessory device 192 may have a diameter/width less than or equal to approximately 6.35 mm (0.25 inches) or less than or equal to approximately 4.2 mm. As discussed above, large-diameter accessory device 192 may be passed through working channel 142 with elevator 126 in a raised configuration. Like small-diameter accessory device 190, large-diameter accessory device 192 may extend through depression 170 of boss 166. As shown in FIGS. 5A-5D, a large-diameter accessory device may have a width/diameter similar to a width/diameter of fenestration 152. Thus, it may not be possible or necessary to lower elevator 126 in order to lock/retain large-diameter accessory device 192 in a configuration in which large diameter accessory device 192 extends approximately parallel to a longitudinal axis of distal tip 120/shaft 18. Fenestration 152 may function similarly to a working channel opening of a conventional distal-viewing medical device in order to constrain large-diameter accessory device 192. A perimeter of fenestration 152 may prevent large-diameter accessory device 192 from moving upwards through the top, open side of channel 162. Large-diameter accessory device 192 may extend approximately parallel to a longitudinal axis of distal tip 120 and/or shaft 18. Features of guide surface 154 may assist in guiding large-diameter accessory device 192 through fenestration 152.

It will be appreciated that elevator 126 may be moved to configurations intermediate of the configurations shown in FIGS. 4A-4D and 5A-5D to lock accessory devices of varying sizes. Alternatively, elevator 126 may be retained in the fully raised configuration of FIGS. 3A-3D, thereby allowing an accessory device to be freely moved proximally/distally (e.g., longitudinally) while being at least partially constrained by a perimeter/circumference of fenestration 152.

Although accessory devices 190, 192 are described as extending approximately parallel to the longitudinal axis of distal tip 120 and/or shaft 18, it will be appreciated that shafts of the accessory devices 190, 192 may be flexible, and those devices thus may passively or actively bend (particularly if the devices are extended distally of distal tip 120 by a large distance). The description of accessory devices 190, 192 as extending approximately parallel to the longitudinal axis of distal tip 120 and/or shaft 18 is intended to particularly apply to a configuration of accessory devices 190, 192 at or near distal tip 120.

Thus, features of elevator 126 and/or channel 162 (including bottom wall 161 that defines a portion of channel 162) may facilitate using medical device 10 as both a forward-viewing medical device and as a side-viewing medical device. The same medical device 10 may be used for EGD and ERCP procedures. For a particular procedure, medical device 10 may be used as only a forward-viewing device or as a side-viewing device. Alternatively, medical device 10 may be used in both manners in a single procedure. As discussed above, distal tip 120 may facilitate dual-locking; an accessory device may be locked in a retroflexed position (or another position where the accessory device extends radially outward) or in a position in which the accessory device extends distally.

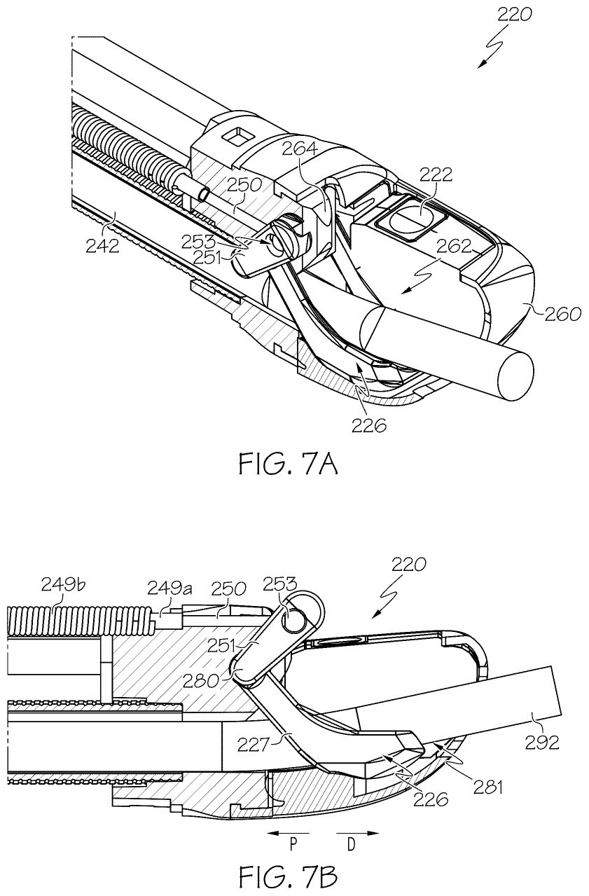

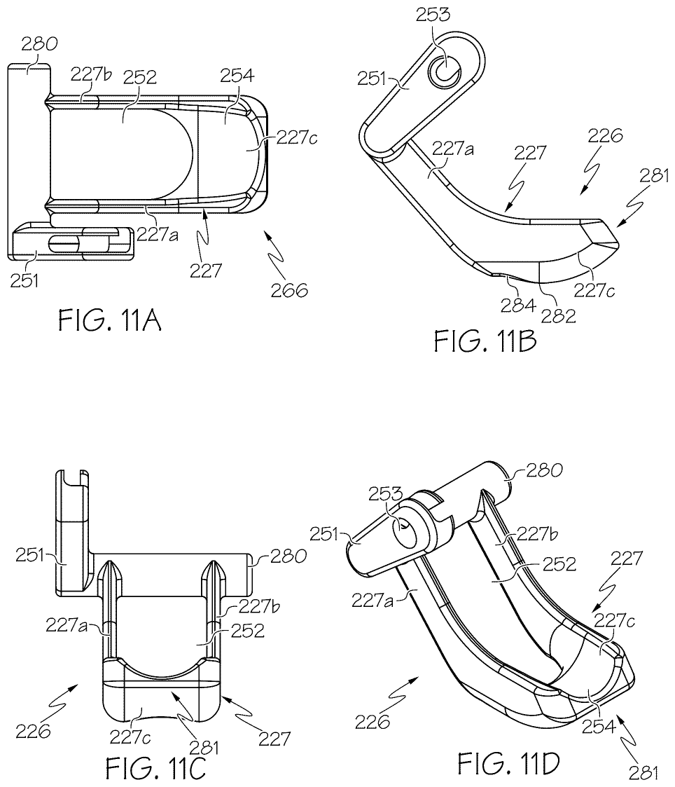

FIGS. 7A-10B depict aspects of an alternative distal tip 220 for use with a medical device 10. Distal tip 220 may have any of the features of distal tips 20, 120, except as described below. Features of distal tips 120 and 220 may be combined in any suitable manner. FIGS. 7A, 8A, 9A, and 10A depict perspective cross-sections of distal tip 220. FIGS. 7B, 8B, 9B, and 10B illustrate side views of cross-sections taken along the same planes of FIGS. 7A, 8A, 9A, and 10A. Certain elements of distal tip 220 (e.g., caps, covers, sheaths, etc.) may be omitted from FIGS. 7A-10B to more clearly illustrate aspects of the disclosure. FIGS. 11A-11D depict aspects of an elevator 226 of distal tip 220. Where feasible, reference numbers of FIGS. 7A-11D add 100 to the reference numbers of FIGS. 2A-6E to denote similar structures.

Distal tip 220 may include a housing 260, having any of the features of housing 160, unless otherwise specified. Housing 260 may define a channel 262, having any of the features of channel 162, unless otherwise specified. Distal tip 220 may include an imaging device 222, having any of the features of imaging device 122.

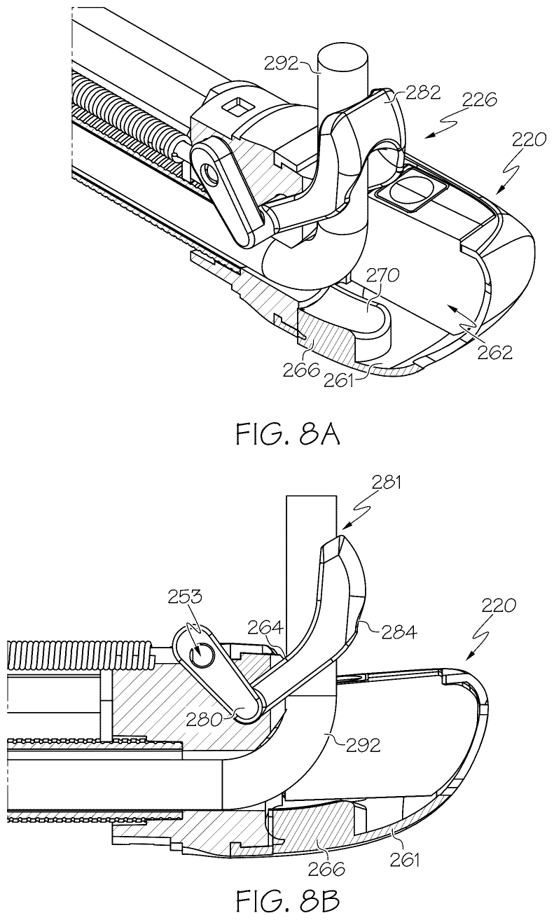

Distal tip 220 may also include an elevator 226. FIGS. 11A-11D depict aspects of elevator 226 separate from distal tip 220. Like elevator 126, elevator 226 may include a fenestration 252. However, whereas elevator 126 pivots about an axle 180 that is below working channel 142 (i.e., closer to bottom wall 161 than working channel 142 is), elevator 226 may be coupled to housing 260 such that it pivots about an axle 280 that is above working channel 242. In other words, axle 280 about which elevator 226 rotates may be positioned on a side of working channel 242 that is closer to imaging device 222 than a bottom wall 261 (see, e.g., FIG. 8A) of housing 260.

Elevator 226 may include a body 227 coupled to an arm 251. As shown in, for example, FIGS. 11A and 11C, a first end of arm 251 may be coupled to axle 280, which may, in turn, be coupled to body 227. Axle 280 may have an approximately cylindrical shape. Body 227 may include two legs 227a, 227b, which extend from axle 280, toward a distal end 281 of elevator 226. A distal portion 227c of body 227 may extend between legs 227a and 227b. Distal portion 227c may have a first, guide surface 254, having any of the properties of guide surface 154. Guide surface 254 may be configured to receive and guide an accessory device along elevator 226. Distal portion 227c also may have a second, back surface 282. Back surface 282 and guide surface 254 may form opposite sides of elevator 226. Guide surface 254 and back surface 282 may be on opposite sides of axle 280, about which elevator 226 rotates.

As shown particularly in FIG. 11A, fenestration 252 may have a proximal side defined by a distal surface of axle 280. Legs 227a, 227b may define lateral sides of fenestration 252. The proximal and lateral sides of fenestration 252 may be approximately straight/linear. A proximal edge of distal portion 227c may define a distal side of fenestration 252. The distal side of fenestration 252 may be curved (e.g., approximately semi-circular shaped).

Arm 251 may extend at an approximately 90 degree angle relative to longitudinal axes of legs 227a, 227b (the longitudinal axes of which may be approximately parallel to one another) and relative to a longitudinal axis of axle 280. An end of arm 251 opposite axle 280 may include an opening 253. Opening 253 may have any suitable shape and may be, for example, circular. As discussed below, opening 253 may receive control elements for rotating elevator 226.

As shown particularly in FIG. 11B, body 227 may have a curved shape. A side profile of distal portion 227c (shown in FIG. 11B) of body may extend at an angle relative to longitudinal axes of legs 227a, 227b. Overall, including arm 251, elevator 226 may have a modified “C” or modified “U” shape when viewed from the side.

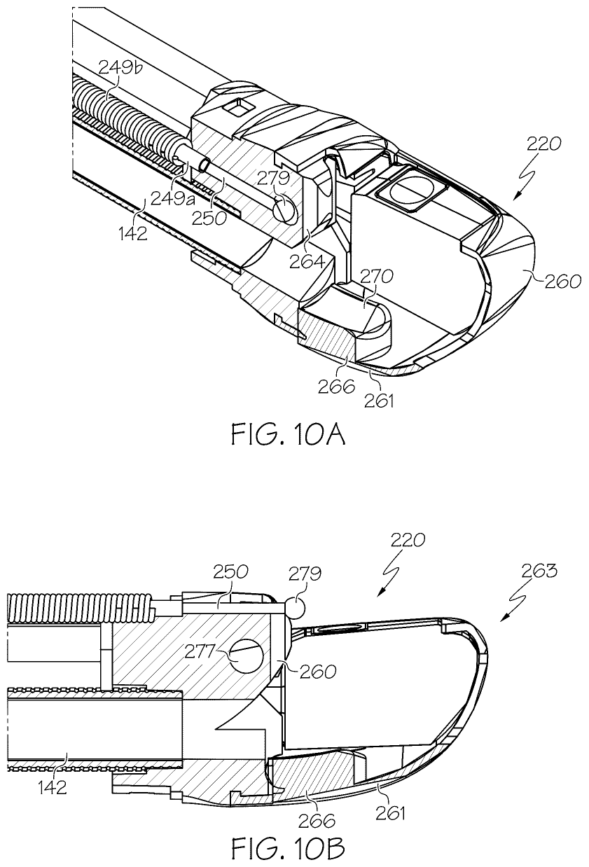

As shown in, for example, FIG. 7B, a control wire 250 may be coupled to arm 251 of elevator 226 via opening 253. Proximal portions of control wire 250 may pass through a sheath 249a and a coil 249b, such that an assembly of control wire 250, sheath 249a, and/or coil 249b is or is similar to a Bowden cable. FIGS. 10A and 10B depict distal tip 220 without elevator 226. As shown in FIGS. 10A and 10B, control wire 250 may have a distal protrusion 279. Distal protrusion 279 may be approximately spherical or may have any other suitable shape. Distal protrusion 279 may be coupled to elevator 226 via opening 253.

As shown in FIG. 10B, hole 277 may be formed in housing 260. Axle 280 of elevator 226 may be received in hole 277 to couple elevator 226 to housing 260. Elevator 226 may be rotatable about axle 280 as control wire 250 moves proximally or distally. Moving control wire 250 distally may lower elevator 226, and moving control wire 250 proximally may raise elevator 226.

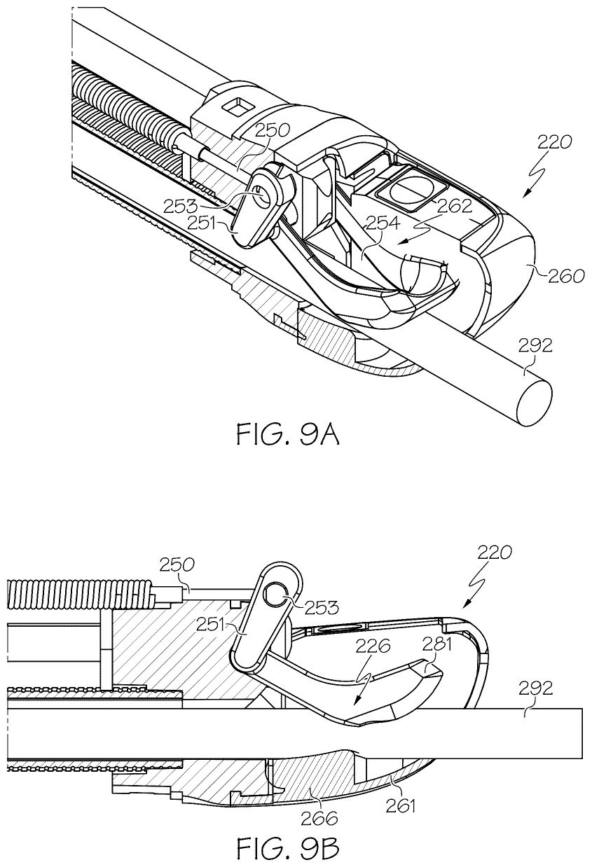

As shown particularly in FIGS. 8A-8B and 10A-10B, a bottom wall 261 of housing 260 may have a boss 266. Boss 266 may have any of the properties of boss 166, unless otherwise stated. Boss 266 may have a shape that complements a shape of fenestration 252. For example, boss 266 may have approximately straight sides and a curved distal end. Boss 266 may have a depression 270 (similar to depression 170) extending longitudinally along boss 266, for receiving and/or interacting with an accessory device 292 (see FIGS. 9A and 9B).

Various configurations and uses of distal tip 220 will now be described, along with methods for using distal tip 220. In an example, device 10 and distal tip 220 may be used as a typical side-viewing medical device (e.g., for an ERCP procedure). Elevator 226 may have a first, lowered configuration, as shown in FIGS. 7A and 7B. As shown in FIGS. 7A and 7B, during an ERCP procedure or other procedure in which side-viewing features of device 10 are to be utilized, an accessory device 292 may be passed through working channel 242 with elevator 226 in the lowered configuration. Although a large-diameter accessory device 292 is depicted, accessory device 292 may alternatively be a small-diameter accessory device 292.

Boss 266 (e.g., a distal portion of boss 266) may be received within fenestration 252 (e.g., within a distal portion of fenestration 252). Boss 266 may serve to provide a smooth transition between channel 262 and elevator 226, so that accessory device 292 is guided onto guide surface 254 instead of exerting a force on elevator 226 and thereby rotating elevator 226. As shown in FIG. 2C, accessory device 292 may rest on a proximal portion of boss 266 and on guide surface 254. A distal, top surface of boss 266 may approximately align with guide surface 254. With boss 266 received within fenestration 252, accessory device 292 may pass along guide surface 254 as with a conventional elevator of a side-viewing scope, until a distal tip of accessory device 292 is distal of distal end 281 of elevator 226.

During an ERCP procedure, the user may raise elevator 226 to a raised position/configuration, as shown in FIG. 8A and 8B, thereby articulating accessory device 292 (e.g., to a retroflexed configuration of the accessory device or another configuration where the accessory device extends radially outward (e.g., approximately 90 degrees outward). In some aspects, accessory device 292 may be locked (retained in a desired position/configuration) between a portion of guide surface 254 (e.g., distal end 281) and a locking surface 264 (see FIGS. 8B and 10B) of housing 260. Locking surface 264 may be at a proximal side of channel 262. Locking surface 264 may have a complementary shape to guide surface 254, such that guide surface 254 may mate with locking surface 264. Additionally or alternatively, locking surface may have an approximately “V” shape. Locking surface 264 may have any features known in the art. In the raised configuration, although boss 266 is no longer positioned within fenestration 252, a distal end of accessory device 292 has already been extended past distal end 281. Thus, fenestration 252 does not interfere with an articulation of accessory device 292 or with locking of accessory device 292 between guide surface 254 and locking surface 264. Therefore, distal tip 220 allows a user to perform a procedure, such as ECRP, as the procedure is conventionally performed with a side-viewing device.

In another example, a user may pass accessory device 292 over elevator 226 with elevator 226 in the lowered configuration/position, as described above and shown in FIGS. 7A and 7B. The user may perform a procedure without raising elevator 226. For example, the user may use medical device 10 as a forward-viewing device for, e.g., an EGD procedure. In such a configuration, accessory device 292 may be free to move (e.g., float) up/down (in a direction of FIG. 7A) through channel 262, including through the open top side of channel 262.

Alternatively, the user may desire to perform a procedure such as an EGD procedure with accessory device 292 in a locked position, unable to freely move upward/downward. In order to do so, before passing accessory device 292 fully through working channel 242, the user may raise elevator 226 to a raised position (similar to that shown in FIGS. 8A and 8B but without accessory device 292 present). The user may then extend accessory device through working channel 242, and through fenestration 252 of elevator 226. Working channel 242 may be in communication with fenestration 252. For example, fenestration 252 may be approximately aligned with working channel 242. Fenestration 252 may be akin to a distal opening of working channel 242 when elevator 226 is in a fully raised configuration. Accessory device 292 may extend distally of a distalmost end 263 (see FIG. 8B) of housing 260 and a distalmost end of channel 262.

As shown in FIGS. 9A and 9B, distal tip 220 may be operative to retain accessory device 292 (either a large-diameter or a small-diameter accessory device) so that accessory device 292 extends distally of distal tip 220 (e.g., approximately parallel to a longitudinal axis of distal tip 220 and/or shaft 18 (FIG. 1A). To lock small-diameter accessory device 290, a user may partially lower elevator 226, such that back surface 282 of elevator 226 and boss 266 engage accessory device 290, as shown in FIGS. 9A-9B. This engagement may such that accessory device 292 extends approximately parallel to a longitudinal axis of distal tip 220/shaft 18 (i.e., lock accessory device 292). An amount or degree that elevator 226 is lowered to lock accessory device 292 may depend upon one or more dimensions or other properties of distal tip 220 and/or a diameter/width of accessory device 292. Accessory device 292 may extend through depression 270 in the locked configuration. Back surface 282 may have a contoured recess 284 may have a shape that mates with/interacts with accessory device 292 so as to engage accessory device 292 between contoured recess 284 and a portion of boss 266, such as depression 270.

Although accessory device 292 is described as extending approximately parallel to the longitudinal axis of distal tip 220 and/or shaft 18, it will be appreciated that a shaft of accessory devices 292 may be flexible, and the device thus may passively or actively bend (particularly if the devices are extended distally of distal tip 220 by a large distance). The description of accessory device 292 as extending approximately parallel to the longitudinal axis of distal tip 220 and/or shaft 18 is intended to particularly apply to a configuration of accessory devices 292 at or near distal tip 220.

Thus, features of elevator 226 and/or channel 262 (including bottom wall 261 that defines a portion of channel 262) may facilitate using medical device 10 as both a forward-viewing medical device and as a side-viewing medical device. The same medical device 10 may be used for EGD and ERCP procedures. For a particular procedure, medical device 10 may be used as only a forward-viewing device or as a side-viewing device. Alternatively, medical device 10 may be used in both manners in a single procedure. As discussed above, distal tip 220 may facilitate dual-locking; an accessory device may be locked in a retroflexed position (or another position where the accessory device extends radially outward) or in a position in which the accessory device extends distally.

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Additionally, a variety of elements from each of the presented embodiments can be combined to achieve a same or similar result as one or more of the disclosed embodiments. Accordingly, the invention is not to be considered as limited by the foregoing description.

We claim:

1. A distal tip of a medical device, comprising:
a housing; and
an elevator, wherein the elevator includes an opening extending from a first surface of the elevator to a second surface of the elevator;
wherein the opening is configured to receive an accessory medical device therethrough, such that the accessory medical device extends distally of a distalmost end of the housing.

2. The distal tip of claim 1, wherein the first surface of the elevator is on a first side of an axis of rotation of the elevator, wherein the second surface of the elevator is on a second side of the axis of rotation, and wherein the first side is opposite the second side.

3. The distal tip of claim 1, wherein, in a raised configuration of the elevator, the opening is configured to receive the accessory medical device therethrough, and wherein, in the raised configuration of the elevator, the first surface is proximal of the second surface.

4. The distal tip of claim 1, wherein the housing includes a boss, and wherein, in a lowered configuration of the elevator, the boss is configured to be received within the opening.

5. The distal tip of claim 4, wherein the second surface of the elevator and the boss are configured to engage the accessory medical device in a configuration in which the accessory medical device is received through the opening, thereby retaining the accessory medical device in a locked configuration, in which the accessory medical device extends approximately parallel to a longitudinal axis of the distal tip.

6. The distal tip of claim 5, wherein the boss includes a depression, wherein the depression is configured to receive the accessory medical device.

7. The distal tip of claim 5, wherein the second surface of the elevator includes a recess, wherein, in the locked configuration, the accessory medical device is received within a recess of the second surface.

8. The distal tip of claim 7, wherein the recess is distal of the opening.

9. The distal tip of claim 1, wherein the opening has a fully enclosed perimeter.

10. The distal tip of claim 8, wherein the opening is approximately circular.

11. The distal tip of claim 1, wherein the distal tip also includes a channel, wherein the elevator is received within the channel.

12. The distal tip of claim 11, wherein the channel has an open distal end.

13. The distal tip of claim 11, wherein the housing defines at least one surface of the channel, and wherein the at least one surface includes a notch at a distal end.

14. The distal tip of claim 11, wherein the housing defines a locking surface at a proximal end of the channel, wherein the locking surface is configured to mate with the first surface of the elevator in a raised configuration of the elevator.

15. The distal tip of claim 1, wherein, in a lowered configuration of the elevator, the distal tip is configured to receive the accessory medical device such that the accessory medical device extends from a proximal end of the elevator to a distal end of the elevator.

16. An elevator of a medical device, the elevator including:
an axle, wherein the elevator is rotatable about the axle, and wherein an axis of rotation of the elevator extends through the axle;
a first surface on a first side of the axis of rotation;
a second surface on a second side of the axis of rotation; and
an opening extending through the elevator, between the first surface and the second surface.

17. The elevator of claim 16, wherein the opening has a fully enclosed perimeter.

18. A method of using a medical device, the method comprising:
inserting an accessory medical device into a working channel of the medical device;
extending the accessory medical device distally to a distalmost end of the medical device, with an elevator of the medical device in a raised configuration; and
using the accessory medical device to perform a medical procedure.

19. The method of claim 18, further comprising, after extending the accessory medical device distally, at least partially lowering the elevator to engage the elevator between a distal surface of the elevator and a surface of a distal tip of the medical device.

20. The method of claim 19, wherein the surface of the distal tip is a surface of a boss extending from a housing of the distal tip.
